(19) United States
(12) Patent Application Publication
(10) Pub. No.: US 2012/0136335 A1
(43) Pub. Date: May 31, 2012

(54) FOCAL MEDICATION TITRATION SYSTEM

(76) Inventor: Jonathan P. Hobbs, Riceville, TN (US)

(21) Appl. No.: 12/956,277

(22) Filed: Nov. 30, 2010

(51) Int. Cl. A61M 37/00

(52) U.S. Cl. 604/503; 604/66

(57) ABSTRACT

A system is provided for implantation into the skull of a patient including a device to detect the onset and/or existence of a neurological condition and deliver a medication to prevent the neurological condition, and/or to improve or sustain normal neurological function. Methods for utilizing the system and device are also provided.

### TECHNICAL FIELD

Aspects of the inventions disclosed herein are directed to systems, devices and methods for establishing a device that is implanted into a portion of the skull for purposes such as delivering therapy in response to a medical condition. The therapy is typically in the form of a locally delivered medication (e.g., altered cerebral spinal fluid concentrations, pharmacological stimulation, chemotherapy and the like) to targeted structures in the brain in a controlled manner, with or without feedback, to maintain or improve normal neurological function.

### BACKGROUND

Neurological Disorders

Neurological disorders are complex, and are typically the result of a cascade of biochemical, electrical, and/or morphological abnormalities. As with any complex disorder, such as epilepsy, cancer, Alzheimer's, or dementia, diagnosis and treatment are challenging. Symptoms may overlap, as is often the case with epileptic seizures.

Epilepsy is a devastating neurological disorder. However, the hallmark symptom of a seizure, abnormal electrodynamics, does not necessarily indicate epilepsy as the primary disorder. Often the underlying pathology is a brain tumor. In fact, many patients present with seizures and their subsequent magnetic resonance image (MRI) scans reveal tissue abnormalities. Depending on the type of tumor and severity of seizure, the clinician may prescribe some combination of chemotherapy, radiotherapy, and/or anti-seizure medication. In cases where the tumor is aggressive, the tumor may be surgically removed. Typically, a seizure focus (region where the abnormal electrodynamics consistently occur) has developed. The focus is often removed with the tumor. Both disorders progress simultaneously in this scenario. The seizures increase in severity as the tumor grows. Current treatments, chemotherapy and anti-seizure medication have limited efficacy and significant side effects to healthy tissue.

Sensing and modulating activity in the brain is a difficult task. The brain is made up of billions of tiny cells packed closely together. All of these cells are encased in a special outer layer of tissue (dura), which is directly under the skull. For all of these reasons, accurate measurement and treatment of neurological disorders are still quite crude. Consequently, progress with fundamental implications in real-time diagnosis and treatment of neurological disorders is of great importance.

Detecting Neurological Activity

Electroencephalography, (EEG) is the measurement of combined electrical potentials produced by networks of nearby neurons. Poor spatial resolution of the EEG, on the order of several centimeters, present challenges for collecting and accurately processing signals. The skull, skin, and hair are insulative barriers that prevent accurate measurement of EEG signals.

Implantable electrodes that lie directly on the surface of the brain or the dura circumvent these signal interference issues. Direct contact with the brain eliminates signal degradation but also poses new problems, principally risk to the patient's health, because the procedure is invasive, as well as expensive, the high expense associated with such a highly technical surgery. Implantable devices have traditionally belonged to one of two categories: chronic, in which a stimulator is typically implanted in the torso, with wires or leads run under the skin to the skull where the electrodes are positioned; and acute, where leads exit the skull at the position of the electrodes and the leads are connected to an external device that records neurological activity. Unfortunately, both of these methods have serious drawbacks. First, the chronically implanted system has long wires that break down with mechanical stress, and which can also act as antennas for ambient electromagnetic signals. Second, the acute system leaves the patient vulnerable to infection, and susceptible to injury, as the prosthetic is protruding and exposed.

Brain Cancer

The cells in the brain (neurons) are unique. Unlike other cell types in the body, there is a limited supply of neurons. Only two areas of the brain have been found to produce new neurons after birth, those are in the dentate gyrus, a structure heavily relied upon for learning and memory, and in the olfactory system, where the sense of smell is generated. Large numbers of new cells outside of these two areas are typically considered cancerous.

Chemotherapy and/or irradiation are typical treatments for brain cancer. The problem with both of these therapies is their limited efficacy and significant side effects. In order for chemotherapy to be effective, toxic levels of the chemotherapy are delivered to all cells in the body effecting normal healthy tissue. An alternative to this is focal delivery of the chemotherapy. Chemotherapy can be delivered locally such as when it is delivered in a slowly dissolving media wafer such as polyanhydride. These wafers have been used for brain tumor treatment. A chemotherapy medication is loaded into the polyanhydride wafer and implanted into the brain. The wafer dissolves and the tumor is treated at the site while other tissue has reduced exposure to the medication. There are at least two drawbacks to wafer chemotherapy: (1) besides pre-formulation, there is little or no control over how the wafer dissolves; and (2) the wafer has a limited supply of medication and cannot be refilled but instead it must be replaced. In the former condition, it would be advantageous to control the rate of medication release as tumors often alter regulation of cellular processes. For example, if the cancer slows down in growth, then rate of medication release could also be decreased. In the latter condition, it would be cost effective and safer to perform the surgery only once and refill the medication instead of performing another surgery.

Implantable Devices

A suitable treatment for neurological disorders would be an implantable, programmable device that delivered therapy to an identified site when needed, and which could be refilled when necessary. Current treatment for neurological disorders requires oral, (sometimes intravenous) administered medication, but a device to deliver this treatment does not exist. Treatment of diseases unrelated to epilepsy or brain tumors require medications that are limited by half-life and stability of physiological concentrations, and require treatment delivery by implanted device. A wearable pump can deliver a stable dose of insulin to a patient. Stable release of insulin is a requirement for insulin deficient patients as fluctuations in levels can result in devastating consequences.

Obesity is often associated with diabetes. Over 90% of people with type 2 diabetes are overweight, and with half of all Americans being overweight, obesity is an epidemic. Appetite-suppressing hormone can be delivered via implanted pump. Melanocortin binds to the Melanocortin receptor in the brain, which results in appetite suppression. Physiologically, the half-life of Melanocortin is so short that infusion has to be delivered at a constant rate which is why oral, and hypodermic delivery are not feasible. These methods of treatment are directed at diseases unrelated to cancer or neurological disorders.

What is needed is a treatment administered to abnormal tissue, whether it be cancerous, putative epileptic foci, degenerative, or other neuropathy, without affecting normal, healthy tissue thereby limiting side effects while increasing, or maintaining efficacy of the therapy. The present novel technology addresses this need.

### BRIEF DRAWING DESCRIPTION

FIG. 1 provides a diagram illustrating one embodiment of an implantable device according to the present disclosure.

FIG. 2 provides a side view of an embodiment of an implantable device according to the present disclosure.

FIG. 3 is a bottom view of an embodiment of an implantable device according to the present disclosure.

FIG. 4 is a schematic of an embodiment of a low voltage amplifier according to the present disclosure.

FIG. 5 is a bottom view of an embodiment of a therapy delivery system according to the present disclosure.

FIG. 6 is a top view of an embodiment of a power source and charging system according to the present disclosure.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

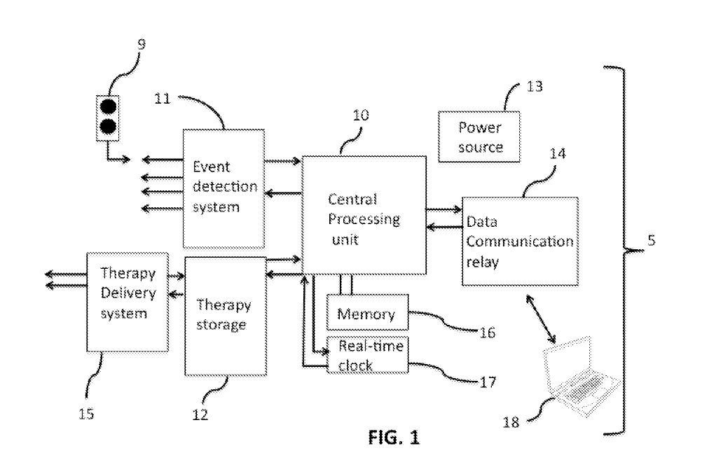

FIG. 1 illustrates the configuration of implantable systems 5 for treating neurological disorders. The device is able to deliver therapy to a region of the brain, such as by program, the detection of a condition, or the like. One typical embodiment implantable system 5 includes a central processing unit (CPU) 10 for carrying out the instructions of the program, performing all necessary transformations of the data, and controlling input and output from an event detection system 11 operationally connected thereto. The event detection system 11 is connected to one or more sensor 9 for activation of therapy storage 12 and delivery of therapy 15. The system 5 is typically powered by a on-board, externally rechargeable power source 13, which also supports memory 16 for input/output and programming instructions, chronologically synchronized by a real-time clock 17, with all instructions writable by data and communications relay 14.

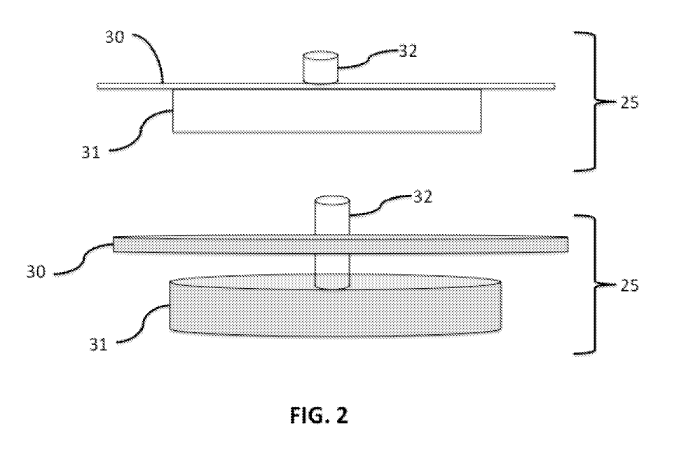

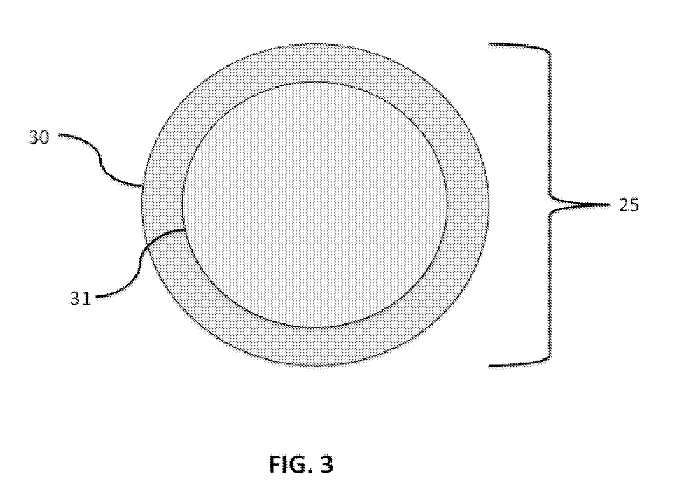

FIGS. 2 & 3 illustrate the outer encasement 25 of the device 5. The encasement 25 typically has three portions, the outer flange 30, the main body 31, and the refill port 32. The refill port 32 extends through the outer flange 30 and into the main body 31 to access the therapy storage 12 (see FIG. 1). The outer flange 30 is typically attachable to a bony or fibrous structure for stability. In this arrangement, the device 5 is detachable from the outer flange 30. As seen in FIG. 3, the flange 30 has the form of an outer ring distal to the main body 31 of the device 5 (see FIG. 1).

In one embodiment the sensors 9 (see FIG. 1) are typically arranged in a grid or linear strip of platinum, stainless steel, or other convenient material typically used as an electrode to sense electromagnetic signals generated by neural tissue. The electrodes are embedded in a flexible or pliable material, typically a silicone or flexible plastic that can conform to the surface of the brain providing contact. Electrodes are typically about 2 to 3 mm in diameter, with exact inter-electrode spacing depending on the particular needs of the patient. Leads connecting the sensors 9 (see FIG. 1) to the event detection system 11 (see FIG. 1) would typically be contact welded to the respective sensors 9 (see FIG. 1).

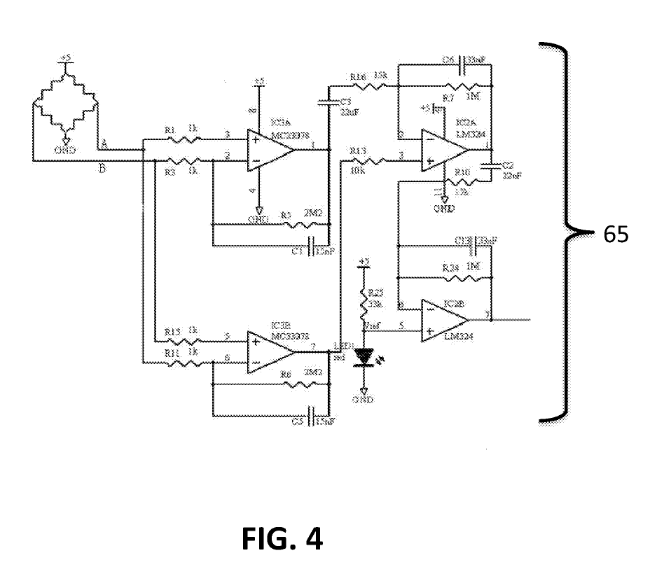

Sensors 9 (see FIG. 1) are typically connected to the event detection system 11 (see FIG. 1). Signals generated by the brain will be microvolt scale and need to be amplified before transmitted to the CPU 10 (see FIG. 1). A typical event detection system 11 (see FIG. 1) amplifies the signals generated by the brain. A typical embodiment of this amplification process can be found in FIG. 4. Typically the microvolt signals generated by the brain will be low frequency. Referring to FIG. 4, the amplifier grouping 65 exhibits the lowest signal to noise ratio at 5 Hz. Typically, the events of interest will be detected by increased amplitude, which is likely to be at or near 5 Hz.

One embodiment of this device 5 (see FIG. 1) includes a programmable microcontroller as a central processing unit 10 (see FIG. 1). A fully programmable microcontroller with 32 KB self-programming flash program memory, 2 KB SRAM, 1024 Bytes EEPROM, 8 Channel 10-bit A/D converter (TQFP/MLF) is typically sufficiently powerful to support the system 5. If memory and clock functions are built into the CPU 10 (see FIG. 1), external memory 16 (see FIG. 1) and a real-time clock 17 (see FIG. 1) are unnecessary. This particular embodiment utilizes a clean and stable power supply, which could be delivered by a 100 mA 5V low-dropout voltage regulator.

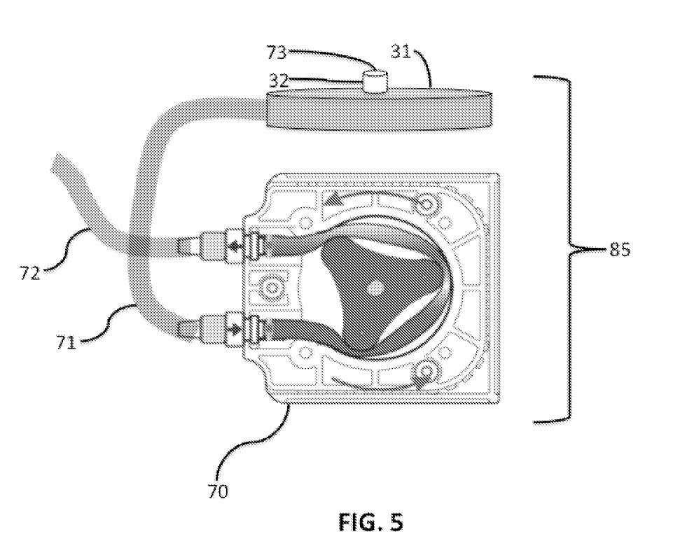

FIG. 5 is a typical embodiment of the therapy system 85 having a miniaturized peristaltic pump 70 attached to the therapy storage 31, typically comprised of stainless steel, plastic or other biocompatible material. Therapy is drawn from the vessel by flexible connector 71 as needed, and delivered by flexible connector 72 to the site in need of therapy. The therapy storage unit 31 (see FIG. 2) will have a membrane 73 that is permeable to hypodermic needles for refilling. Delivery of therapy 85 is accurate, and precisely controlled by the CPU 10 (see FIG. 1).

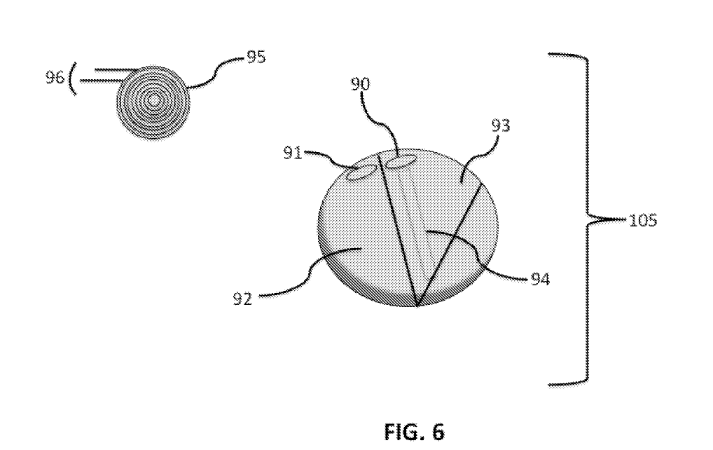

A typical power source for an embodiment of this device is shown in FIG. 6. This power source 105 provides 12V electricity to the device. An embodiment of the power source is circular in shape having a negative tap 90, a positive tap 91 connected to a positive core 92 which is separated by a barrier from the negative core 93 and negative electrode 94. The power source 105 is typically charged by induction coil 95 connected by charging leads 96. The materials used in the power source could be Lithium ion, Nickel Cadmium, or any other known convenient element that holds an electrical potential.

In one embodiment of the disclosed device, data relay 14 (see FIG. 1) is transmitted by radio frequency. The data relay 14 (see FIG. 1) can operate in the upper GHz range receiving instructions from a base station 18 (see FIG. 1).

The system 5 (see FIG. 1) delivers therapy in the form of a medication for treating a neurological disorder and or disease. The system 5 (see FIG. 1) is implantable and provides a means to deliver therapy focally to abnormal tissue.

Epilepsy

The present novel technology provides medication focally in a closed-loop manner. In one embodiment, the system 5 (see FIG. 1) is able to detect the onset of aberrant electrical field potentials that may indicate the condition of an oncoming seizure. The condition being met, therapy is typically delivered in the form of cerebral spinal fluid with elevated magnesium CSFEM, anti-seizure medication, non-traditional seizure therapy by excitatory, and inhibitory agonists and antagonists or the like.

Cerebral Spinal Fluid with Elevated Magnesium

Normal cerebral spinal fluid, (CSF) contains a concentration of essential ions. The bulk of these ions are sodium. CSF is actively transported across the blood brain barrier by the choroid plexus at a rate of about 0.5 ml/min or about 720 ml per day. CSF flows through the ventricles throughout the brain where it exits either through the cranial nerve roots or the arachnoid villi. Physiological concentrations of CSF are as follows in mM: NaCl 125, KCl 3, NaH2PO4*H2O 1.25, NaHCO3 26, MgSO4*7H2O 1.2, CaCl2*2H2O 2, D-glucose 10. Neural tissue is highly sensitive to changes in these concentrations of ions or protein. The serum protein level of CSF is 100 times lower than that of blood. For these reasons, small changes in concentrations of CSF or protein rapidly alter neurological activity.

In one embodiment, device 5 (see FIG. 1) will, upon detection of a condition, increase the local level of magnesium in the CSF. The “steady state” of neurons are highly dependent on the equilibrium potential of the ions. Realistically, neurons are always in flux, and the “steady state” refers to a range of values at which neurons are not exhibiting an action potential, or strongly depolarized.

The neurons in the cortex are highly interconnected, as is evident from the cellular organization, into six distinct layers. Output from the cortex to the rest of the body is primarily transmitted from layers 3, 5 and 6. The local electric field of layers 3, 5, & 6 is dominated by pyramidal cell interactions. The pyramidal cell will not exhibit action potentials if a magnesium ion is bound to the pore of the N-Methyl D-Aspartate receptors (NMDA). Increasing the local concentration of magnesium blocks the action potential of these cells and will arrest the onset of the seizure safely and effectively.

Anti-Seizure Therapy

In one embodiment, this device 5 (see FIG. 1) will upon detection of a condition, indicating the onset of a seizure, deliver a therapy. The condition being met, therapy in the form of medication is delivered focally through microinjection. Anti-seizure medications work through many different pathways to prevent the onset of seizure, but most decrease excitation. Most medication work by inactivating voltage gated sodium channels making them unavailable to pass sodium ions, and consequently the neuron is unable to spike. Table 1 provides a partial listing of anti-seizure medications proposed as medicants.

TABLE 1
Commonly prescribed anti-seizure medications:

1-[(2,6-difluorophenyl)methyl]-1H-1,2,3-triazole-4 carboxamide,
5H-dibenzo[b,f]azepine-5-carboxamide,
7-Chloro-1,5-dihydro-1-methyl-5-phenyl-1,5-benzodiazepine-2,4(3H)-
dione,
5-(2-Chlorophenyl)-1,3-dihydro-7-nitro-1,4--benzodiazepin-2-one,
Sodium 2-propylpentanoate,
7-chloro-1-methyl-5-phenyl-3H-1,4-benzodiazepin-2-one,
sodium 5,5-diphenyl-2,4-imidazolidinedione,
3-ethyl-3-methylpyrrolidine-2,5-dione,
3-carbamoyloxy-2-phenylpropyl,
8-chloro-5-methyl-1-phenyl-1,5-benzodiazepine-2,4-dione,
2-[1-(aminomethyl)cyclohexyl]acetic acid,
(3R)-1-[4,4-bis(3-methylthiophen-2-yl)but-3-enyl]piperidine-3-carboxylic
acid],
(2R)-2-(2-oxopyrrolidin-1-yl)butanamide,
5-(2-chlorophenyl)-7-nitro-2,3-dihydro-1,4-benzodiazepin-2-one,
3,5-diamino-6-(2,3-dichlorophenyl)-as-triazine,
6-(2,3-dichlorophenyl)-1,2,4-triazine-3,5-diamine,
(−)-(S)-α-ethyl-2-oxo-1-pyrrolidine acetamide,
5-ethyl-5-phenyl-1,3-diazinane-2,4,6-trione,
(3S)-3-(aminomethyl)-5-methylhexanoic acid,
5-ethyl-5-phenyl-1,3-diazinane-4,6-dione,
2-[1-(aminomethyl)cyclohexyl]acetic acid,
5-oxo-6H-benzo[b][1]benzazepine-11-carboxamide,
5-ethyl-5-phenyl-1,3-diazinane-2,4,6-trione,
[(3R,3aS,6aR)-2,3,3a,4,5,6a-hexahydrofuro[5,4-b]furan-3-yl] N-[(2S,3R)-
4-[(4-aminophenyl)sulfonyl-(2-methylpropyl)amino]-3-hydroxy-1-
phenylbutan-2-yl]carbamate,
5,5-di(phenyl)imidazolidine-2,4-dione,
4-aminohex-5-enoic acid,
benzo[b][1]benzazepine-11-carboxamide,
(3R)-1-[4,4-bis(3-methylthiophen-2-yl)but-3-enyl]piperidine-3-carboxylic
acid,
2,3:4,5-Bis-O-(1-methylethylidene)-beta-D-fructopyranose sulfamate,
5-oxo-6H-benzo[b][1]benzazepine-11-carboxamide,
2-propylpentanoic acid,
(R)-2-acetamido-N-benzyl-3-methoxypropionamide,
3-ethyl-3-methylpyrrolidine-2,5-dione,
1,2-benzoxazol-3-ylmethanesulfonamide.

Drug Side Effects

Long-term use of these drugs can have serious side effects. Severe allergic reactions (rash; hives; itching; difficulty breathing; tightness in the chest; swelling of the mouth, face, lips, or tongue); bone pain; butterfly-shaped rash on the face; clumsiness or unsteadiness; confusion; dark urine; delirium; fast, slow, or irregular heartbeat; high blood sugar (flushing; fruit-like breath odor; increased thirst, hunger, or urination; rapid breathing); mental or mood changes; numbness or tingling of the hands or feet; pain, swelling, or redness at the injection site; red, swollen, blistered, or peeling skin; severe or persistent dizziness or drowsiness; signs of infection (eg, chills, fever, sore throat); slurred speech; stomach pain; swollen lymph nodes; swollen or tender gums; tremor; unusual bruising or bleeding; unusual eye movements; unusual muscle movements; yellowing of the skin or eyes.

An advantage of this embodiment is the focal administration of the medication. Each time the condition is met for therapy, the medication is applied locally, increasing its efficacy, and decreasing risk of side effects for the patients. This method also decreases the risk of increased tolerance to the therapy, as administration occurs only when necessary, not continuously.

Anti-Cancer

In one embodiment, device 5 (see FIG. 1) administers targeted therapy to the abnormal brain region. Medication therapy is delivered focally through microinjection. The medication therapy being a form of but not limited to chemotherapy with anti-cancer properties, and anti-seizure medications for simultaneous treatment of coexisting conditions.

Chemotherapy

Brain tumors are one of the most deadly forms of cancer. As noted above, the tumor may be found secondary to the initial condition of seizures or neuropathy. The survival rate for glioblastoma, one of the most commonly occurring brain cancers, is 1 year from diagnosis. During this year the patient typically undergoes heavy doses of chemotherapy and/or irradiation to combat the cancer. The problem with these treatments is that they target healthy tissue along with cancerous tissue. This problem could be addressed by a method that targeted abnormal tissue and did not treat, or treated negligibly the healthy tissue. Table 2 provides a partial list of chemotherapy drugs that are proposed medicants.

TABLE 2
Commonly prescribed chemotherapy drugs:

2-amino-4,6-dimethyl-3-oxo-N,N′-bis[7,11,14-trimethyl-2,5,9,12,15-pentaoxo-3,10-
di(propan-2-yl)-8-oxa-1,4,11,14-tetrazabicyclo[14.3.0]nonadecan-6-
yl]phenoxazine-1,9-dicarboxamide,
(7S,9S)-7-[(2R,4S,5S,6S)-4-amino-5-hydroxy-6-methyloxan-2-yl]oxy-6,9,11-
trihydroxy-9-(2-hydroxyacetyl)-4-methoxy-8,10-dihydro-7H-tetracene-5,12-dione,
2-amino-3-[4-[bis(2-chloroethyl)amino]phenyl]propanoic acid,
4-amino-1-[(2R,3S,4S,5R)-3,4-dihydroxy-5-(hydroxymethyl)oxolan-2-yl]pyrimidin-
2-one,
2,4,5-trioxa-1,3-diarsabicyclo[1.1.1]pentane,
Humanized anti-VEGF antibody,
1,3-bis(2-chloroethyl)-1-nitrosourea,
4-methylsulfonyloxybutyl methanesulfonate,
cis-diammine(cyclobutane-1,1-dicarboxylate-O,O′)platinum(II),
1-(2-chloroethyl)-3-cyclohexyl-1-nitrosourea,
azane; dichloroplatinum,
2H-1,3,2-Oxazaphosphorin-2-amine, N,3-bis(2-chloroethyl)-tetrahydro-, 2-oxide,
(7S,9S)-9-acetyl-7-[(2R,4S,5S,6S)-4-amino-5-hydroxy-6-methyloxan-2-yl]oxy-
6,9,11-trihydroxy-4-methoxy-8,10-dihydro-7H-tetracene-5,12-dione,
(5Z)-5-(dimethylaminohydrazinylidene)imidazole-4-carboxamide,
5-fluoro-1H-pyrimidine-2,4-dione,
(3R,4S,5S,6R,7R,9R,11R,12R,13S,14R)-6-[(2S,3R,4S,6R)-4-dimethylamino-3-
hacyclotetradecane-2,10-dione,
[(2R,3S,4S,5R)-5-(6-amino-2-fluoropurin-9-yl)-3,4-dihydroxyoxolan-2-yl]methyl
dihydrogen phosphate,
4-amino-1-[(2R,4R,5R)-3,3-difluoro-4-hydroxy-5-(hydroxymethyl)oxolan-2-
yl]pyrimidin-2-one,
Humanized anti-HER2 antibody,
hydroxyurea,
7S,9S)-9-acetyl-7-[(2R,4S,5S,6S)-4-amino-5-hydroxy-6-methyloxan-2-yl]oxy-6,9,11-
trihydroxy-8,10-dihydro-7H-tetracene-5,12-dione,
N,N-bis(2-chloroethyl)-1,3,2-oxazaphosphinan-2-amine 2-oxide
irinotecan hydrochloride,
N-[3-chloro-4-[(3-fluorophenyl)methoxy]phenyl]-6-[5-[(2-
methylsulfonylethylamino)methyl]furan-2-yl]quinazolin-4-amine,
(2R,3S,5R)-5-(6-amino-2-chloropurin-9-yl)-2-(hydroxymethyl)oxolan-3-ol,
(2S)-2-[[4-[(2,4-diaminopteridin-6-yl)methylmethylamino]benzoyl] amino]
pentanedioic acid,
(1S)-5-Deoxy-1-C-[(2S,3S)-7-[[2,6-dideoxy-3-O-(2,6-dideoxy-β-D-arabino-
hexopyranosyl)-β-D-arabino-hexopyranosyl]oxy]-3-[(O-2,6-dideoxy-3-C-methyl-β-
D-ribo-hexopyranosyl-(1→3)-O-2,6-dideoxy-β-D-lyxo-hexopyranosyl-(1→3)-2,6-
dideoxy-β-D-arabino-hexopyranosyl)oxy]-1,2,3,4-tetrahydro-5,10-dihydroxy-6-
methyl-4-oxo-2-anthracenyl]-1-O-methyl-D-threo-2-pentulose,
6-Amino-1,1a,2,8,8a,8b-hexahydro-8-(hydroxymethyl)-8a-methoxy-5-methyl-
azirino[2′,3′:3,4]pyrrolo[1,2-a]indole-4,7-dione carbamate (ester),
1,4-dihydroxy-5,8-bis[2-(2-hydroxyethylamino)ethylamino]anthracene-9,10-dione,
3′,4′-didehydro-4′-deoxy-C′-norvincaleukoblastine [R—(R*,R*)-2,3-
dihydroxybutanedioate,
Bis(2-chloroethyl) methylamine,
Nitrogen mustard,
Chimeric murine/human monoclonal anti-CD20 antibody,
2-amino-3,7-dihydropurine-6-thione,
5 beta,20-Epoxy-1,2a,4,7 beta,10 beta,13 alpha-hexahydroxytax-11-en-9-one 4,10-
diacetate 2-benzoate 13-ester with (2R,3S)—N-benzoyl-3-phenylisoserine,
(2R,3S)—N-carboxy-3-phenylisoserine,N-tert-butyl ester, 13-ester with 5β-20-epoxy-
1,2α,4,7β,10β,13α-hexahydroxytax-11-en-9-one 4-acetate 2-benzoate, trihydrate,
(S)-10-[(dimethylamino)methyl]-4-ethyl-4,9-dihydroxy-1H pyrano[3′,4′:6,7]
indolizino[1,2-b]quinoline-3,14(4H,12H)-dione monohydrochloride,
methyl (1R,9R,10S,11R,12R,19R)-11-(acetyloxy)-12-ethyl-4-[(13S,15S,17S)-17-
ethyl-17-hydroxy-13-(methoxycarbonyl)-1,11-diazatetracyclo[13.3.1.04,12.05,10]
nonadeca-4(12),5,7,9-tetraen-13-yl]-8-formyl-10-hydroxy-5-methoxy-8,16-
diazapentacyclo[10.6.1.01,9.02,7.016,19]nonadeca-2,4,6,13-tetraene-10-
carboxylate,
4′-demethyl-epipodophyllotoxin 9-[4,6-O—(R)-ethylidene-beta-D-glucopyranoside],
4′-(dihydrogen phosphate),
pentyl[1-(3,4-dihydroxy-5-methyl-tetrahydrofuran-2-yl)-5-fluoro-2-oxo-1H
pyrimidin-4-yl]aminomethanoate.

Apoptosis

Programmed cell death or apoptosis is induced by chemotherapy. Evidence has shown that inhibition of the transcription factor NF-_{K}B leads to enhanced apoptosis in cancer cell lines. It is impossible for cellular machinery to make new cells if the information for making them cannot be read. While this is a valuable treatment for cancer, there are caveats. Namely, all cells, not just cancer cells use NF-_{K}B as a transcription factor so all cells are indiscriminately targeted, albeit different rates for apoptosis. In one embodiment, the device 5 (see FIG. 1) is programmed to deliver targeted chemotherapy consisting of a compound that inhibits NF-_{K}B delivered to the site of the cancerous tumor, reducing side effects to normal healthy tissue.

Nuclear Receptor

The most common type of brain tumor is glioblastoma. As with any type of cancer, the wrong type of cell replicates in the wrong region of the body, which disturbs normal function of that area. In glioblastoma, cancerous tumor stem cells proliferate and destroy sensitive neural networks that enable normal brain function. Tumor cell types can be targeted and prevented from replicating. As tumor cells replicate, the levels of nuclear factor expression in vivo rise. A sample of tumor can be assayed, and the appropriate nuclear factor ligand can be prescribed to arrest the replication of the brain tumor stem cells. The phylogenetic tree of nuclear factors can be found in table 3 referred to herein as the nuclear factor superfamily. In one embodiment, the device can be programmed to deliver targeted therapy consisting of nuclear factor superfamily ligand delivered to the site of the cancerous tumor, reducing side effects to normal healthy tissue, and arresting tumor stem cell replication.

TABLE 3
Nuclear factor superfamily:

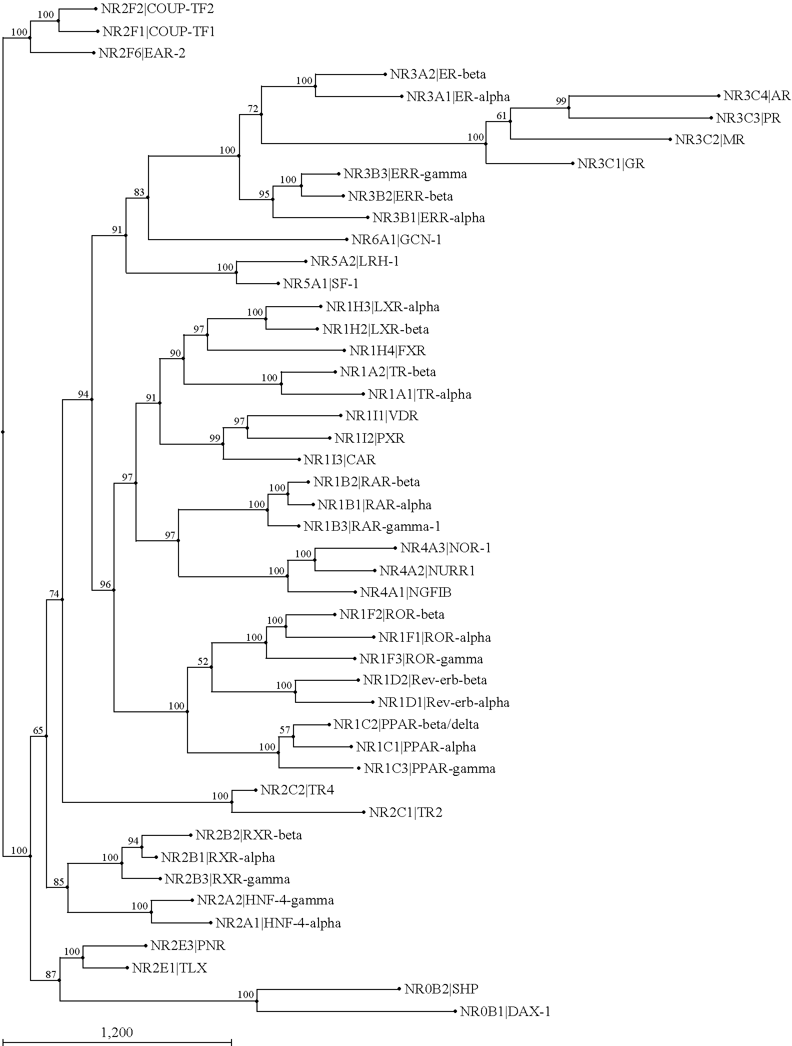

A novel method is presented in this disclosure for treating an individual subject to chronic neurological abnormality. The method of treatment includes implanting a device 5 (see FIG. 1) under the scalp. The device 5 (see FIG. 1) has a detection system 11 (see FIG. 1) operationally connected to at least one sensor 9 (see FIG. 1) capable of detecting electromagnetic fields generated by the brain. Upon detection of a condition in the signal the CPU 10 (see FIG. 1) which is operationally connected to the therapy storage system 12 (see FIG. 1) activates the therapy delivery system 15 (see FIG. 1) to deliver a predetermined amount of medication to a predetermined area of the brain. Programming of the device is performed through RF data relay 14 (see FIG. 1) by base station 18 (see FIG. 1). The medication is typically in the form of an anti seizure medication, or additional magnesium ions. The focal delivery of the various medications provided herein is novel and provides the advantages provided herein. Anti seizure medication can be taken orally, but the major draw back to this method is that all cells in the body are affected by the medication. Oral administration of magnesium ions for neurological therapy is impossible, as filtering will occur before interstitial fluid crosses the blood brain barrier.

Focal treatment of a brain tumor is a novel method of this disclosure. Chemotherapy is typically taken orally or administered intravenously which suffers from the same drawbacks as every cell is affected in the body. Upon diagnosis of a tumor, the device 5 (see FIG. 1) delivers a therapy to arrest the growth of the tumor. The therapy storage system 12 (see FIG. 1) is activated by therapy delivery system 15 (see FIG. 1), to deliver a chemotherapy agent. This novel delivery system of a chemotherapy agent provides more effective treatment at a limited dosage thus substantially lowering unwanted side effects.

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following claims are desired to be protected.

1. An implantable device for treating a neurological episode comprising:
(a) a detection system having at least one sensor capable of detecting electromagnetic field signals;
(b) a processing system operationally coupled to the detection system for receiving and processing signals; and
(c) a therapy system operationally connected to the processing system;
wherein, the processing system:
(i) identifies signals received which are indicative of the neurological episode;
(ii) determines a medication need; and
(iii) activates the therapy system upon determination of a medication need; and
wherein, the therapy system is configured to deliver predetermined amounts of medication to a predetermined location at predetermined intervals upon the determination of a medication need.

2. The device of claim 1, wherein the device is configured to deliver a medication including an anti-seizure medication for treatment of epilepsy.

3. The device of claim 2, wherein the anti-seizure medication includes magnesium.

4. A method for treating an individual subject to chronic neurological abnormality comprising:
(a) implanting a treatment device under the subject's scalp;
(b) detecting neurological electromagnetic field signals indicative of a neurological episode;
(c) providing a therapy to interrupt the neurological episode;
(d) obtaining further neurological electromagnetic field signals indicative of a resolution of the neurological episode; and
(e) interrupting the therapy upon obtaining signals indicative of the resolution of the episode;
wherein the steps of detecting, providing, obtaining, and interrupting are carried out by the device, and wherein the medication further comprises an anti-seizure medication.

5. The method of claim 4, wherein the anti-seizure medication includes magnesium ion.

6. The method of claim 4, wherein the anti-seizure medication is selected from the group consisting of:
1-[(2,6-difluorophenyl)methyl]-1H-1,2,3-triazole-4 carboxamide,
5H-dibenzo[b,f]azepine-5-carboxamide,
7-Chloro-1,5-dihydro-1-methyl-5-phenyl-1,5-benzodiazepine-2,4(3H)-dione,
5-(2-Chlorophenyl)-1,3-dihydro-7-nitro-1,4-benzodiazepin-2-one,
Sodium 2-propylpentanoate,
7-chloro-1-methyl-5-phenyl-3H-1,4-benzodiazepin-2-one,
sodium 5,5-diphenyl-2,4-imidazolidinedione,
3-ethyl-3-methylpyrrolidine-2,5-dione,
3-carbamoyloxy-2-phenylpropyl,
8-chloro-5-methyl-1-phenyl-1,5-benzodiazepine-2,4-dione,
2-[1-(aminomethyl)cyclohexyl]acetic acid,
(3R)-1-[4,4-bis(3-methylthiophen-2-yl)but-3-enyl]piperidine-3-carboxylic acid],
(2R)-2-(2-oxopyrrolidin-1-yl)butanamide,
5-(2-chlorophenyl)-7-nitro-2,3-dihydro-1,4-benzodiazepin-2-one,
3,5-diamino-6-(2,3-dichlorophenyl)-as-triazine,
6-(2,3-dichlorophenyl)-1,2,4-triazine-3,5-diamine,
(−)-(S)-α-ethyl-2-oxo-1-pyrrolidine acetamide,
5-ethyl-5-phenyl-1,3-diazinane-2,4,6-trione,
(3S)-3-(aminomethyl)-5-methylhexanoic acid,
5-ethyl-5-phenyl-1,3-diazinane-4,6-dione,
2-[1-(aminomethyl)cyclohexyl]acetic acid,
5-oxo-6H-benzo[b][1]benzazepine-11-carboxamide,
5-ethyl-5-phenyl-1,3-diazinane-2,4,6-trione,
[(3R,3aS,6aR)-2,3,3a,4,5,6a-hexahydrofuro[5,4-b]furan-3-yl]N-[(2S,3R)-4-[(4-aminophenyl)sulfonyl-(2-methylpropyl)amino]-3-hydroxy-1-phenylbutan-2-yl]carbamate,
5,5-di(phenyl)imidazolidine-2,4-dione,
4-aminohex-5-enoic acid,
benzo[b][1]benzazepine-11-carboxamide,
(3R)-1-[4,4-bis(3-methylthiophen-2-yl)but-3-enyl]piperidine-3-carboxylic acid,
2,3:4,5-Bis-O-(1-methylethylidene)-beta-D-fructopyranose sulfamate,
5-oxo-6H-benzo[b][1]benzazepine-11-carboxamide,
2-propylpentanoic acid,
(R)-2-acetamido-N-benzyl-3-methoxypropionamide,
3-ethyl-3-methylpyrrolidine-2,5-dione,
1,2-benzoxazol-3-ylmethanesulfonamide, and
a combination thereof.

7. A method for providing chemotherapy comprising:
(a) identifying a tumor and its location; and
(b) delivering, a therapy, proximate the tumor, to interrupt the tumor's growth;
wherein the therapy is delivered by a subdural device comprising:
(i) a processing system for determining a need for therapy; and
(ii) a therapy deliver system operationally connected to the processing system for delivering predetermined amounts of medication to predetermined sites;
wherein, upon determining the need for therapy, the processing system activates the therapy delivery system to provide the therapy which includes a medication.

8. The method of claim 7, wherein delivering, a therapy, proximate the tumor, is carried out according to a predetermined manner controlled by the processing system.

9. The method of claim 7, wherein delivering, a therapy, proximate the tumor, is carried out according to instructions currently determined and communicated to the therapy delivery system through the processing system.

10. The method of claim 7, wherein delivering, a therapy, proximate the tumor involves delivering a chemotherapy agent.

11. The method of claim 10, wherein the chemotherapy agent is selected form the group consisting of:
2-amino-4,6-dimethyl-3-oxo-N,N′-bis[7,11,14-trimethyl-2,5,9,12,15-pentaoxo-3,10-di(propan-2-yl)-8-oxa-1,4,11,14-tetrazabicyclo[14.3.0]nonadecan-6-yl]phenoxazine-1,9-dicarboxamide,
(7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione,
2-amino-3-[4-[bis(2-chloroethyl)amino]phenyl]propanoic acid,
4-amino-1-[(2R,3S,4S,5R)-3,4-dihydroxy-5-(hydroxymethyl)oxolan-2-yl]pyrimidin-2-one,
2,4,5-trioxa-1,3-diarsabicyclo[1.1.1]pentane,
Humanized anti-VEGF antibody,
1,3-bis(2-chloroethyl)-1-nitrosourea,
4-methylsulfonyloxybutyl methanesulfonate,
cis-diammine(cyclobutane-1,1-dicarboxylate-O,O′)platinum(II),
1-(2-chloroethyl)-3-cyclohexyl-1-nitrosourea,
azane; dichloroplatinum,
2H-1,3,2-Oxazaphosphorin-2-amine, N,3-bis(2-chloroethyl)-tetrahydro-, 2-oxide,
(7S,9S)-9-acetyl-7-[(2R,4S,5S,6S)-4-amino-5-hydroxy-6-methyloxan-2-yl]oxy-6,9,11-trihydroxy-4-methoxy-8,10-dihydro-7H-tetracene-5,12-dione,
(5Z)-5-(dimethylaminohydrazinylidene)imidazole-4-carboxamide,
5-fluoro-1H-pyrimidine-2,4-dione,
(3R,4S,5S,6R,7R,9R,11R,12R,13S,14R)-6-[(2S,3R,4S,6R)-4-dimethylamino-3-hacyclotetradecane-2,10-dione,
[(2R,3S,4S,5R)-5-(6-amino-2-fluoropurin-9-yl)-3,4-dihydroxyoxolan-2-yl]methyl dihydrogen phosphate,
4-amino-1-[(2R,4R,5R)-3,3-difluoro-4-hydroxy-5-(hydroxymethyl)oxolan-2-yl]pyrimidin-2-one,
Humanized anti-HER2 antibody,
hydroxyurea,
7S,9S)-9-acetyl-7-[(2R,4S,5S,6S)-4-amino-5-hydroxy-6-methyloxan-2-yl]oxy-6,9,11-trihydroxy-8,10-dihydro-7H-tetracene-5,12-dione,
N,N-bis(2-chloroethyl)-1,3,2-oxazaphosphinan-2-amine 2-oxide irinotecan hydrochloride,
N-[3-chloro-4-[(3-fluorophenyl)methoxy]phenyl]-6-[5-[(2-methylsulfonylethylamino)methyl]furan-2-yl]quinazolin-4-amine,
(2R,3S,5R)-5-(6-amino-2-chloropurin-9-yl)-2-(hydroxymethyl)oxolan-3-ol,
(2S)-2-[[4-[(2,4-diaminopteridin-6-yl)methylmethylamino]benzoyl]amino]pentanedioic acid,
(1S)-5-Deoxy-1-C-[(2S,3S)-7-[[2,6-dideoxy-3-O-(2,6-dideoxy-β-D-arabino-hexopyranosyl)-β-D-arabino-hexopyranosyl]oxy]-3-[(O-2,6-dideoxy-3-C-methyl-β-D-ribo-hexopyranosyl-(1→3)-O-2,6-dideoxy-β-D-lyxo-hexopyranosyl-(1→3)-2,6-dideoxy-β-D-arabino-hexopyranosyl)oxy]-1,2,3,4-tetrahydro-5,10-dihydroxy-6-methyl-4-oxo-2-anthracenyl]-1-O-methyl-D-threo-2-pentulose,
6-Amino-1,1a,2,8,8a,8b-hexahydro-8-(hydroxymethyl)-8a-methoxy-5-methyl-azirino[2′,3′:3,4]pyrrolo[1,2-a]indole-4,7-dione carbamate (ester),
1,4-dihydroxy-5,8-bis[2-(2-hydroxyethylamino)ethylamino]anthracene-9,10-dione,
3′,4′-didehydro-4′-deoxy-C′-norvincaleukoblastine[R—(R*,R*)-2,3-dihydroxybutanedioate,
Bis(2-chloroethyl)methylamine,
Nitrogen mustard,
Chimeric murine/human monoclonal anti-CD20 antibody,
2-amino-3,7-dihydropurine-6-thione,
5 beta,20-Epoxy-1,2a,4,7 beta,10 beta,13 alpha-hexahydroxytax-11-en-9-one 4,10-diacetate 2-benzoate 13-ester with (2R,3S)—N-benzoyl-3-phenylisoserine,
(2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate trihydrate,
(S)-10-[(dimethylamino)methyl]-4-ethyl-4,9-dihydroxy-1H pyrano[3′,4′:6,7]indolizino[1,2-b]quinoline-3,14(4H,12H)-dione monohydrochloride,
dimethyl(2β,3β,4β,5α,12β,19α)-15-[(5S,9S)-5-ethyl-5-hydroxy-9-(methoxycarbonyl)-1,4,5,6,7,8,9,10-octahydro-2H-3,7-methanoazacycloundecino[5,4-b]indol-9-yl]-3-hydroxy-16-methoxy-1-methyl-6,7-didehydroaspidospermidine-3,4-dicarboxylate,
methyl(1R,9R,10S,11R,12R,19R)-11-(acetyloxy)-12-ethyl-4-[(13S,15S,17S)-17-ethyl-17-hydroxy-13-(methoxycarbonyl)-1,11-diazatetracyclo[13.3.1.04,12.05,10]nonadeca-4(12),5,7,9-tetraen-13-yl]-8-formyl-10-hydroxy-5-methoxy-8,16diazapentacyclo[10.6.1.01,9.02,7.016,19]nonadeca-2,4,6,13-tetraene-10-carboxylate,
4′-demethyl-epipodophyllotoxin 9-[4,6-O—(R)-ethylidene-beta-D-glucopyranoside], 4′-(dihydrogen phosphate), and
pentyl[1-(3,4-dihydroxy-5-methyl-tetrahydrofuran-2-yl)-5-fluoro-2-oxo-1H pyrimidin-4-yl]aminomethanoate.

12. The method of claim 7, wherein delivering, a therapy, proximate the tumor involves delivering a medication including a nuclear factor ligand.
